(19) United States
(12) Patent Application Publication
(10) Pub. No.: US 2016/0106302 A1
(43) Pub. Date: Apr. 21, 2016

(54) ENDOSCOPE

(71) Applicant: OLYMPUS WINTER & IBE GMBH, Hamburg (DE)

(72) Inventor: Martin WIETERS, Hamburg (DE)

(73) Assignee: OLYMPUS WINTER & IBE GMBH, Hamburg (DE)

(21) Appl. No.: 14/985,997

(22) Filed: Dec. 31, 2015

Related U.S. Application Data
(63) Continuation of application No. PCT/EP2014/001655, filed on Jun. 18, 2014.

(30) Foreign Application Priority Data
Jul. 2, 2013 (DE) 102013212854.1

(51) Int. Cl. A61B 1/00; A61B 1/005

(52) U.S. Cl. CPC A61B 1/00158; A61B 1/00165; A61B 1/0056; A61B 1/0057

(57) ABSTRACT

An endoscope including: a shaft having an outer tube and an inner tube, the inner tube being arranged inside the outer tube; and a contact-free magnetic coupling. The contact-free magnetic coupling including: an outer coupling part arranged concentrically with the outer tube, the outer coupling part being arranged outside the outer tube in a radial direction; and an inner coupling part arranged concentrically with the inner tube, the inner coupling part being arranged inside the inner tube in the radial direction; and one or more of light-guiding fibers and electrical conductors are disposed longitudinally within a gap between the outer tube and the inner tube. Where the inner coupling part is configured to be one or more of translatorily and rotationally moved by means of a magnetic operative connection between the outer coupling part and the inner coupling part; and the magnetic operative connection through the gap is rotationally non-symmetric.

### CROSS-REFERENCE TO RELATED APPLICATION

The present application is a continuation of PCT/EP2014/001655 filed on Jun. 18, 2014, which is based upon and claims the benefit to DE 10 2013 212 854.1 filed on Jul. 2, 2013, the entire contents of each of which are incorporated herein by reference.

### BACKGROUND

1. Field

The present application relates to an endoscope, such as a video endoscope, comprising a shaft having an outer tube and an inner tube arranged inside the outer tube and a contact-free magnetic coupling which comprises an outer coupling part that is arranged concentrically with the outer tube outside the outer tube and an inner coupling part that is arranged concentrically with the inner tube inside the inner tube, wherein the inner coupling part can be translatorily and/or rotationally moved by means of a magnetic operative connection between the outer coupling part and the inner coupling part, wherein, in a gap between the outer tube and the inner tube, light-guiding fibers and/or electrical conductors are guided through.

2. Prior Art

Endoscopes are known, which have movable components that are located in a hermetically sealed area of the endoscope. Magnetic couplings for moving the components are known, which have an inner coupling part and an outer coupling part, which are connected to the components, wherein the outer coupling part is connected to an external handle of the endoscope for example. Other components of the endoscope, for example optical fibers of a video endoscope, have to be guided in part between outer and inner coupling parts.

A corresponding generic contact-free magnetic coupling of an endoscope is known from German patent application no. 10 2011 078 969.3 by the applicant. The magnetic coupling comprises an outer coupling part and an inner coupling part, wherein the inner coupling part is arranged concentrically inside the outer coupling part, wherein a ring-shaped gap remains between the coupling parts. The outer coupling part and the inner coupling part each comprise an annular body, wherein the annular body of the outer coupling part is arranged between lateral anchor disks, which together result in a substantially U-shaped cross-section open toward the inside and/or the annular body of the inner coupling part is arranged between lateral anchor disks, which together result in a substantially U-shaped cross-section open toward the outside, wherein the annular body of the outer coupling part and/or of the inner coupling part comprises an axially magnetized ring magnet. This configuration allows a translatory transfer of movement. In addition, the anchor disks of both coupling parts have, on each of their respective surfaces adjacent to the gap between the coupling parts, a mutually corresponding structuring in a peripheral direction, which has pole shoe segments that transmit a rotational movement.

Also known as stators and rotors, these coupling parts are in many cases separated by two coaxial tubes, which enclose the ring gap between outer and inner coupling parts. Bundles of optical fibers and/or electrical conductors are guided through this annular gap. The area of the gap not passed through by the bundle of optical fibers is usually filled with air.

The torques or forces, which are transferable by such magnetic couplings, depend on the strength of the magnetic operative connection. These depend on the strength of the magnets used and their distance from each other. In practice, the transferable torques and forces are therefore limited.

FIG. 1 shows a contact-free magnetic coupling 1′ of a video endoscope, which is known from the state of the art, in a radial sectional view.

The endoscope 1′ comprises a shaft formed as a double shaft with an outer tube 12 and an inner tube 14, of which the longitudinal extension is respectively aligned transverse to the plane of the view. Inside the inner tube 14 is located a hermetically sealed area 16. In this case the inner tube 14 or the outer tube 12 for example is an integral part of a hermetic barrier between the hermetically sealed area 16 and the surrounding area.

The endoscope 1 is formed as a video endoscope 1 and has movable optical components at the distal end. These optical components (not shown) are located inside the hermetically sealed area 16 and are connected to an inner coupling part 26.

The outer tube 12 is enclosed by an outer coupling part 20 which is connected to a handle (not shown) for example. The outer coupling part 20 and the inner coupling part 26 are formed as coupling parts of a magnetic coupling, wherein a movement of the handle can be transferred to the optics by means of magnetic operative connection between the outer coupling part 20 and the inner coupling part 26.

In the example shown, the outer coupling part 20 comprises a ring magnet 22 which is arranged between two anchor disks 24, wherein, in the view in FIG. 1, the second anchor disk is covered by the anchor disk 24 shown. The inner coupling part 26 consists of a magnetic, such as a ferromagnetic, material for example.

The inner tube 14 is concentrically arranged inside the outer tube 12. A ring gap between the outer tube 12 and the inner tube 14 is thereby created, in which a fiber bundle of optical fibers is guided. The fibers of the fiber bundle 40 are used for example to guide light from an external light source from the proximal end of the endoscope 1 to the distal end of the endoscope 1 and illuminate an observation area there. Since the fibers have a low magnetic permeability, the ring gap is rotationally symmetrical in magnetic terms.

The anchor disks 24 and the inner coupling part 26 have anchors that face each other in pairs respectively, which are arranged at equal intervals on the outer coupling part 20 or respectively on the inner coupling part 26. In the example shown the outer coupling part 20 and the inner coupling part 26 each have four anchors. Magnetic field lines run between the anchors of an anchor pair, or respectively magnetic forces take effect, which bring about a magnetic operative connection between the outer coupling part 20 and the inner coupling part 26.

Translatorial and rotational torques are transferable from the outer coupling part 20 to the inner coupling part 26 by means of this magnetic operative connection. The inner coupling part 26 can be movable and/or rotatable along the longitudinal axis inside the hermetically sealed area 16.

If the mechanical forces affecting the outer coupling part 20 and the inner coupling part 26, for example driving forces on the outer coupling part 20 and frictional forces on the inner coupling part 26, exceed the magnetic forces between the anchors, the outer coupling part 20 and the inner coupling part 26 rotate or move against each other, whereby the magnetic operative connection is separated or released. The transferable torque M is also understood to be the maximum torque of the outer coupling part 20 affecting the inner coupling part 26, with which the magnetic operative connection is not released, due to the forces and counterforces having an effect between the outer coupling part 20 and the inner coupling part 26.

Since the bundle of light-guiding fibers 40 is compact, as is usual in the state of the art, a large gap clearance prevails, so that the magnetic force is limited by the dead space of the air-filled volume 42. The presence of light-guiding fibers does not effectively break the magnetic rotational symmetry, either.

### SUMMARY

Proceeding from this state of the art, an object is to increase the torques and forces transferable into a hermetic area of an endoscope.

Such object is achieved by means of an endoscope, such as, a video endoscope, comprising a shaft having an outer tube and an inner tube that is arranged inside the outer tube and a contact-free magnetic coupling which comprises an outer coupling part that is arranged concentrically with the outer tube outside the outer tube and an inner coupling part that is arranged concentrically with the inner tube inside the inner tube, wherein the inner coupling part can be translatorily and/or rotationally moved by means of a magnetic operative connection between the outer coupling part and the inner coupling part, wherein, in a gap between the outer tube and the inner tube, light-guiding fibers and/or electrical conductors are guided through, characterized in that a magnetic rotational symmetry of the gap is broken.

In the context of the present application, a magnetic rotational symmetry is understood to be a discrete or continuous rotational symmetry. In the case of a continuous magnetic rotational symmetry the magnetic field in the gap has a constant strength in a circumferential direction. A rotation through any angle whatsoever always leads to the same distribution of the magnetic field. A discrete rotational symmetry is a twofold, threefold etc. rotational symmetry for example. For example, with a fourfold rotational symmetry, the magnetic field is the same again after a rotation through 90°. In the context of the invention a broken magnetic rotational symmetry of the gap means that the gap has neither a continuous nor a discrete rotational symmetry for magnetic fields, regardless of how magnetic fields are created outside the gap. Only after a rotation of the circumferential gap through 360° is a magnetic field in the gap provided with the same configuration in terms of the spatial distribution of the permeability in the gap.

The present application is based on the concept that a limiting factor for limiting the transferable magnetic forces is the size of the gap between outer and inner coupling parts. The transferable torque or respectively the transferable translatory force of the magnetic coupling decreases exponentially with the size of this distance. Due to the gap needed owing to the light-guiding fibers and/or electrical conductors to be guided through, the effectiveness of the coupling is weakened, so that the transfer of force may no longer be sufficient in the given installation area.

In the context of the present application, a translatory movement can be a shifting or translation along a longitudinal extension of the tube, whereas a rotational movement can be a turning or rotation around a rotational axis aligned along the longitudinal extension of the tube.

The light-guiding fibers and/or electrical conductors can be laid as a compact bundle. This requires a large gap measurement of the ring-shaped gap to accommodate this bundle. The rest of the ring gap is filled with air and thus forms a type of “dead space,” which is no use to the endoscope and reduces the effectiveness of the magnetic coupling.

With the breaking of the magnetic rotational symmetry, this dead space is minimized in magnetic terms. The maximum gap width weakening the magnetism is only necessary for the area through which the light-guiding fibers and/or electrical conductors are fed. In the remaining area of the gap, measures are taken to break the magnetic rotational symmetry and thus reduce the weakening of the magnetic field. With the breaking of the rotational symmetry, the effectiveness of the magnetic coupling is therefore increased again.

In an embodiment of the endoscope the magnetic rotational symmetry of the gap is broken by geometric means, by the inner coupling part being arranged eccentrically inside the outer coupling part. An eccentric arrangement is understood to be an arrangement in which the central rotational axes of outer and inner coupling parts are moved in parallel yet laterally to each other. Therefore the coupling parts are not arranged concentrically with each other.

Due to the eccentric arrangement there is a very short distance between the coupling parts in sections in a peripheral direction of the tube, whereby dead space is reduced and a strong magnetic operative connection is guaranteed between the coupling parts, with correspondingly high transferable torques. By means of this measure the transferable torques become dependent on the positional angle of outer and inner coupling parts relative to any fixed point on the tube. Surprisingly, it has become apparent that the transferable torques or forces for all positional angles are higher than with a symmetrical or concentric arrangement of otherwise unchanged coupling parts. The area gained can, for example, be used to make the endoscope or endoscope components still smaller.

An advantage consists in the transferable torques being improved, while the strength of the magnets used for the magnetic operative connection is kept the same. This prevents overly strong magnets leading to undesired interactions with other components of the endoscope or additional sensitive devices in the surrounding area. Conversely, the present application discloses embodiments that enable the use of weaker magnets relative to known magnetic couplings, with comparable transferable torques.

The outer coupling part can have a discrete or continuous rotational symmetry, wherein the inner coupling part is arranged eccentrically with a symmetry axis of the outer coupling part. A strong magnetic operative connection with correspondingly high transferable torques is thereby guaranteed over a wide range of angles of rotation of the magnetic coupling.

The light-guiding fibers and/or electrical conductors can be arranged as a bundle in an area of a maximum distance between inner and outer tubes.

For example, in the case of several bundles of different thicknesses or one bundle that can be formed to a limited extent in the cross-section, an eccentric configuration of outer and inner coupling parts can also be used, wherein the thickest bundle is laid in the area of the maximum distance and thinner bundles or thinner offshoots or areas of a bundle are laid in one or both tapering parts of the gap. In this way, with a combination of equalizing and eccentric arrangement, the dead space is further minimized.

By means of these measures of uniform distribution or equalization a shorter average distance between the coupling parts is enabled, whereby the magnetic operative connection is strengthened and transferable torques increased.

In an embodiment, which can be used independently or can be used additionally with the previously described embodiments, the light-guiding fibers and/or electrical conductors do not entirely fill the gap, wherein, in at least one part of the gap not filled by the bundle, a filling material having a magnetic permeability between 1.5 and 70, such as between 5 and 11, is arranged. The magnetic permeability μᵣ of the material is, according to the invention, greater than a magnetic permeability of air.

Since the part of the gap, which is passed through by the light-guiding fibers and/or electrical conductors, has a low magnetic permeability and the rest a high magnetic permeability, the magnetic rotational symmetry of the gap is also broken by this measure. The material with high magnetic permeability guides the magnetic field through the gap substantially less weakening than air. In this way an improved permeability of the gap for the magnetic fields running between the coupling parts or a better penetration of the gap with the magnetic fields is achieved relative to an air filled gap. The magnetic operative connection between the coupling parts is thereby strengthened and the transferable torques increased. This measure also reduces the air volume or respectively the dead space in the gap, since the volume now supports the transfer of the magnetic forces through the filling material.

The material can comprise a synthetic material mixed with magnetic, such as soft magnetic, particles. In this context, the feature “magnetic” also means magnetizable and “soft magnetic” means paramagnetic or magnetizable with little remanence.

The light-guiding fibers and/or electrical conductors can be guided through the gap as a bundle that does not entirely enclose the inner tube. In this case an area exists, which can be filled by the filling material.

The filling material advantageously substantially entirely fills the part of the gap free of the light-guiding fibers and/or electrical conductors.

In another embodiment, at least a part of the light-guiding fibers and/or electrical conductors are guided through the filling material or through an opening in the filling material. In this way dead space is further reduced. For this, the filling material can be poured into the gap for example, after the fibers and/or conductors have been laid.

In another embodiment with magnetic couplings with translational stroke, the filling material can extend over an entire length of a translational stroke of the contact-free magnetic coupling in the longitudinal direction of the endoscope shaft.

Additional features will become apparent from the description of embodiments, together with the claims and the attached drawings. Embodiments can fulfill individual features or a combination of several features.

### BRIEF DESCRIPTION OF THE DRAWINGS

Without restricting the general idea of the invention, the invention is described below on the basis of exemplary embodiments with reference to the drawings, whereby reference is explicitly made to the drawings with regard to all the details, which are not described in more detail in the text. In the figures:

FIG. 1 is a schematic illustration of a radial sectional view of a contact-free magnetic coupling of an endoscope, which is known from the state of the art;

FIG. 2 is a schematic illustration of a first embodiment of a radial sectional view of an endoscope;

FIG. 3 schematically shows the transferable torque of the magnetic coupling of the endoscope in FIG. 2, which is dependent on the positional angle;

FIG. 4A schematically shows a first angle position of the magnetic coupling for FIG. 3;

FIG. 4B schematically shows a further angle position of the magnetic coupling for FIG. 3;

FIG. 5 schematically shows a radial sectional view of an endoscope according to a second embodiment.

In the drawings the same and/or similar members or parts are given the same reference numbers respectively, so that a further presentation is dispensed with.

### DETAILED DESCRIPTION

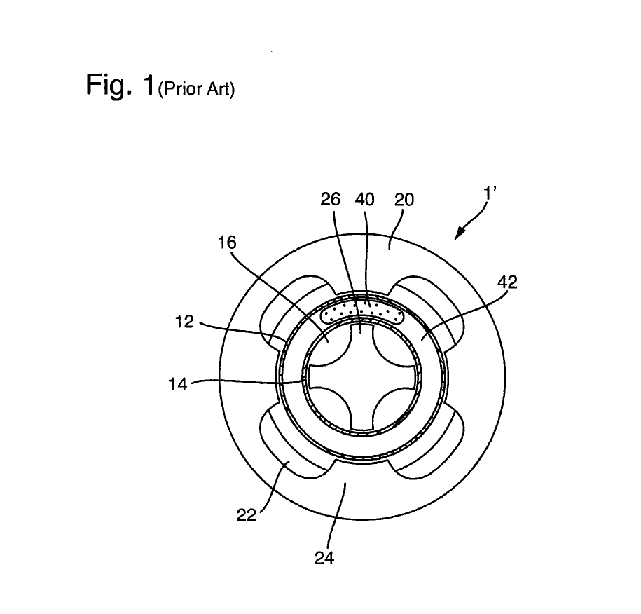

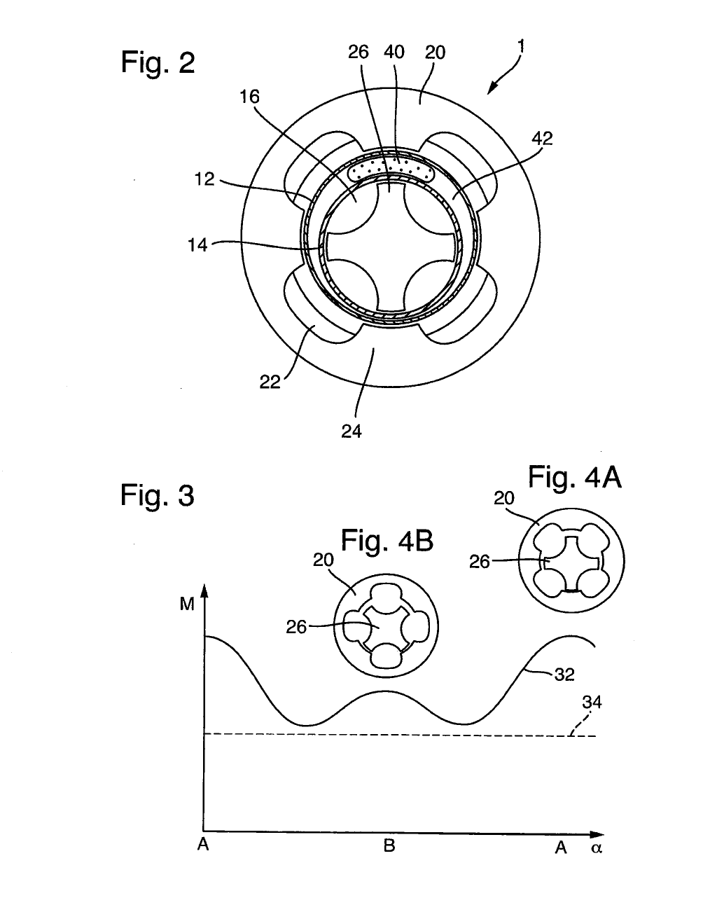

A first embodiment is shown in FIG. 2. In contrast to the magnetic coupling 1′ according to FIG. 1, in the magnetic coupling 1 according to FIG. 2, the inner coupling part 26 is arranged eccentrically in the outer coupling part 20. Further, the outer coupling part 20 is arranged concentrically with the outer tube 12, and the inner coupling part 26 is arranged concentrically with the inner tube 14. Accordingly, the inner coupling part 26 is respectively arranged eccentrically with the outer tube 12 and with the outer coupling part 20, and the outer coupling part 20 is respectively arranged eccentrically with the inner coupling part 26 and the inner tube 14. This results in a simple mechanical construction with broken magnetic rotational symmetry of the gap, with which, the movable outer coupling part 20 is supported on the outer tube 12 and the movable inner coupling part 26 is supported on the inner tube 14, while the outer tube 12 and the inner tube 14 are located as stationary relative to each other.

Although the dimensions of the cross-section of the bundle of light-guiding fibers 40 are unchanged in comparison with FIG. 1, the air-filled volume 42 in FIG. 2 has been substantially reduced relative to FIG. 1, due to the geometric change alone. The mean gap measurement has also decreased thereby.

Due to the eccentric arrangement of outer coupling part 20 and inner coupling part 26, the transferable torque M depends on the angle position or respectively the positional angle of outer coupling part 20 and inner coupling part 26 relative to any fixed point on the stationary tubes 12, 14 of the endoscope. This angle dependence is schematically shown in FIG. 3.

FIG. 3 shows the transferable torque M on the vertical axis and, on the horizontal axis, the positional angle α of the magnetic coupling 1 with outer coupling part 20 and inner coupling part 26. Here, a range of one period of the positional angle α is shown, wherein a period is given through 360°/N or respectively 2π/N, wherein N indicates the number of the respective anchors of the coupling parts 20, 26.

The angle range begins and ends with the angle position A, shown in FIG. 4A, in which an anchor each of the outer coupling part 20 and the inner coupling part 26, respectively, are arranged at minimal distance from each other. In contrast, in the angle position B shown in FIG. 4B, the outer coupling part 20 and the inner coupling part 26 are rotated by half a period, in the example shown with four anchors correspondingly through 45° or π/2, relative to the angle position A.

The course of the maximum transferable torque M dependent on the positional angle α is shown in the characteristic curve 32. The transferable torque M is maximum in position A, decreases as the positional angle α grows, passes through a minimum and reaches an interim maximum in the position B.

By way of comparison, the transferable torque M is shown by a dotted line for the characteristic curve 34, in the case the coupling parts 20, 26 are arranged concentrically with each other with an otherwise unchanged geometry of the magnetic coupling, with regard to e.g., the dimensions of the coupling parts 20, 26 and/or the strength of the magnets used. This characteristic curve 34 runs below the eccentric characteristic curve 32. Hence, with eccentric arrangement of outer coupling part 20 and inner coupling part 26, the transferable torque M, i.e. the course of the characteristic curve 32, is greater for all the positional angles α other than the transferable torque of the concentric arrangement, i.e. the course of the characteristic curve 34. This is a result of the transferable forces exponentially depending on the distance between the pole shoes. Hence pole shoes that have moved closer to each other contribute disproportionately, while a further distancing of pole shoes that are at a distance from each other entails less loss.

A magnetically rotationally symmetrical configuration as in FIG. 1 would lead to a constant characteristic curve which would still be below the characteristic curve 34.

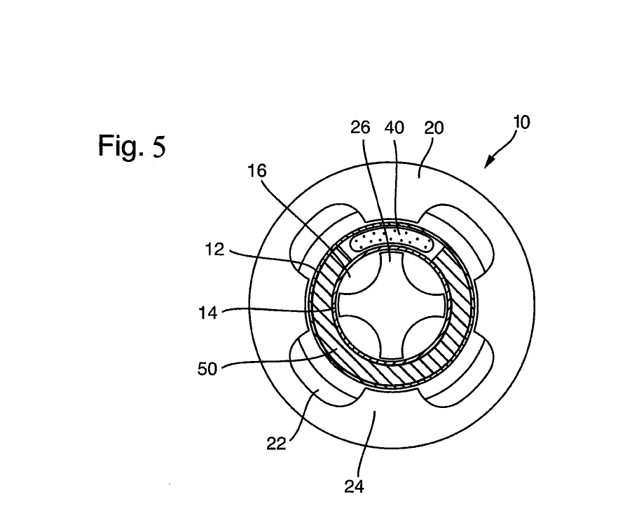

A radial sectional view of a second embodiment of an endoscope 10 is shown in FIG. 5, wherein the sectional plane corresponds to those in FIGS. 1 and 2.

The endoscope 10 has a double shaft having an outer tube 12, an inner tube 14 and a hermetically sealed area 16 inside the inner tube 14. As in the known example in FIG. 1, a bundle of optical fibers 40 is arranged in the gap between outer tube 12 and inner tube 14, wherein the bundle has a compact cross-sectional area and only fills a small part of the gap. However, in addition, a filling material 50 is located in the remaining gap between outer tube 12 and inner tube 14, which comprises, for example, a synthetic material with soft magnetic particles. The material 50 has a magnetic permeability μᵣ which is greater than the magnetic permeability of air and is between 5 and 11 for example. This material in turn breaks the magnetic rotational symmetry of the gap and reduces the dead space in the gap.

The weakening of the magnetic fields between the anchors of outer coupling part 20 and inner coupling part 26 is thereby reduced in comparison to an air-filled gap of the same dimensions, so that the magnetic operative connection between otherwise unchanged outer coupling part 20 and unchanged inner coupling part 26 is strengthened and higher torques can be transferred.

With each of the exemplary embodiments in FIGS. 2 and 5, dead volume is reduced and the transferable forces are increased. Moreover, the air-filled volumes 42 of the eccentric magnetic coupling 1 according to FIG. 2, which have already been reduced, can be filled, with a corresponding filling material 50 according to FIG. 5.

All the features mentioned, also the features which can be inferred from the drawings alone, also individual features, which are disclosed in combination with other features, are regarded as substantial individually and in combination. Embodiments can also be fulfilled by individual features or a combination of several features.

### REFERENCE LIST

1′ Magnetic coupling Of the Prior Art
1 Magnetic Coupling of a First Embodiment
10 Magnetic Coupling of a Second Embodiment
12 Outer tube
14 Inner tube
16 Hermetic area
20 Outer coupling part
22 Ring magnet
24 Anchor disk
26 Inner coupling part
32, 34 Characteristic curve
40 Light-guiding fibers
42 Air-filled volume
50 Filling material
A, B Angle configurations
M Transferable torque
α Angle of rotation

What is claimed is:

1. An endoscope comprising:
a shaft having an outer tube and an inner tube, the inner tube being arranged inside the outer tube; and
a contact-free magnetic coupling comprising:
an outer coupling part arranged concentrically with the outer tube, the outer coupling part being arranged outside the outer tube in a radial direction; and
an inner coupling part arranged concentrically with the inner tube, the inner coupling part being arranged inside the inner tube in the radial direction; and
one or more of light-guiding fibers and electrical conductors are disposed longitudinally within a gap between the outer tube and the inner tube; wherein
the inner coupling part is configured to be one or more of translatorily and rotationally moved by means of a magnetic operative connection between the outer coupling part and the inner coupling part; and
the magnetic operative connection through the gap is rotationally non-symmetric.

2. The endoscope according to claim 1, wherein the inner coupling part is eccentrically arranged inside the outer coupling part.

3. The endoscope according to claim 2, wherein the one or more light-guiding fibers and electrical conductors are disposed as a bundle in an area of a maximum distance between inner tube and outer tube.

4. The endoscope according to claim 1, wherein the one or more light-guiding fibers and electrical conductors do not entirely fill the gap, the endoscope further comprising a filling material having a magnetic permeability between 1.5 and 70 disposed at least partially in a portion of the gap in which the one or more of light-guiding fibers and electrical conductors are not disposed.

5. The endoscope of claim 4, wherein the magnetic permeability is between 5 and 11.

6. The endoscope according to claim 4, wherein the filling material comprises a synthetic material, which is mixed with magnetic particles.

7. The endoscope according to claim 4, wherein the one or more light-guiding fibers and electrical conductors are guided through the gap as a bundle.

8. The endoscope according to claim 4, wherein the filling material substantially entirely fills the portion of the gap which is free of the one or more light-guiding fibers and electrical conductors.

9. The endoscope according to claim 4, wherein at least a portion of the one or more light-guiding fibers and/or electrical conductors are guided through an opening in the filling material.
